(19) United States
(12) Patent Application Publication
(10) Pub. No.: US 2023/0270676 A1
(43) Pub. Date: Aug. 31, 2023

(54) RAPAMYCIN (RAPA) FORMULATION AND PREPARATION METHOD THEREOF

(71) Applicant: Pengke YAN, Guangzhou (CN)

(72) Inventor: Pengke YAN, Guangzhou (CN)

(21) Appl. No.: 18/016,865

(22) PCT Filed: May 31, 2021
(86) PCT No.: PCT/CN2021/097343

§ 371 Date: Jan. 19, 2023

(30) Foreign Application Priority Data
Sep. 30, 2020 (CN) 202011065631.3

(51) Int. Cl. A61K 9/127; A61K 31/436; A61K 47/24

(52) U.S. Cl. CPC A61K 9/1277; A61K 31/436; A61K 47/24

(57) ABSTRACT

A rapamycin (RAPA) liposome formulation and a preparation method thereof are provided. The RAPA formulation includes (in parts by weight): RAPA: 1 to 100 parts; phospholipid: 1 to 2,000 parts; and stabilizer: 0.01 to 100 parts. The preparation method includes: mixing and dissolving RAPA, a phospholipid, and a stabilizer in an organic phase solvent to obtain an organic phase mixed solution; preparing an initial emulsion solution by adding the organic phase mixed solution dropwise to an aqueous phase solvent, and stirring at room temperature to obtain the initial emulsion solution; and conducting lyophilization by homogenizing the initial emulsion solution, adding a lyophilization protective agent, mixing, and filtering a resulting mixture through a microporous filter membrane for sterilization to obtain the RAPA formulation of a liposome-lyophilized powder.

### CROSS REFERENCE TO THE RELATED APPLICATIONS

This application is the national phase entry of International Application No. PCT/CN2021/097343, filed on May 31, 2021, which is based upon and claims priority to Chinese Patent Application No. 202011065631.3, filed on Sep. 30, 2020, the entire contents of which are incorporated herein by reference.

### TECHNICAL FIELD

The present disclosure relates to a rapamycin (RAPA) formulation and a preparation method thereof and belongs to the technical field of medicine.

### BACKGROUND

Cancerous tumors have become the number one killer. Although therapeutic methods for cancerous tumors emerge one after another, survival conditions of most patients have not been greatly improved. Among the various therapeutic methods for cancerous tumors, chemotherapy is still the most common option. Although chemotherapeutic drugs are widely used, chemotherapeutic drugs exhibit uncertain therapeutic effects on solid tumors. This is fundamentally because the traditional chemotherapeutic drug cannot reach an effective therapeutic concentration or cannot work for sufficient time at a tumor site and will indiscriminately kill normal cells, resulting in a variety of side effects and toxic effects. The efficacy of a chemotherapeutic drug depends not only on the sensitivity of the drug but also on the action time of the drug at a tumor site and an accumulated concentration of the drug at a tumor site. Therefore, the targeted application of a chemotherapeutic drug has become a hot spot and challenging point in the research of chemotherapy.

RAPA is a powerful immunosuppressant with low toxicity, which works by binding to a corresponding immunophilin RMBP to inhibit the phases G0 and G1 in a cell cycle and prevent the transition from the phase G1 to the phase S; RAPA is widely used in transplant surgery. In addition to immunosuppression, RAPA has an anti-tumor effect, which can inhibit the growth of tumor cells of kidney cancer, lymphoma, lung cancer, liver cancer, breast cancer, neuroendocrine carcinoma, and gastric cancer, in a concentration-dependent manner. Since 2007, two derivatives of RAPA, temsirolimus and everolimus, have been developed for cancer treatment, and the research and application of RAPA in tumor treatment have been increasing. The application of RAPA alone or in combination with another drug exhibits significant anti-tumor effects both in vitro and in vivo. By inhibiting the mammalian target of RAPA (mTOR) receptor and affecting various signaling pathways for transduction, RAPA plays a variety of effects such as anti-angiogenesis, cell cycle blockade, and apoptosis promotion, which have an impact on the proliferation, invasion, and metastasis of a tumor. However, RAPA is a hydrophobic drug and thus cannot be directly injected, and RAPA has very low bioavailability in vivo and thus easily loses efficacy before reaching a disease site.

### SUMMARY

To overcome the deficiencies of the prior art, a first objective of the present disclosure is to provide a RAPA formulation. The RAPA formulation may have prominent affinity and targetability for tumor cells, make RAPA concentrate in tumor cells by targeting tumor cells, and improve an uptake rate of RAPA by the tumor tissue, thus leading to the apoptosis of tumor cells, thereby treating the tumor.

A second objective of the present disclosure is to provide a preparation method of the RAPA formulation that produces a RAPA formulation of a liposome-lyophilized powder.

A third objective of the present disclosure is to provide a preparation method of the RAPA formulation that produces a RAPA formulation of a fat emulsion.

The first objective of the present disclosure may be achieved through the following technical solutions: A RAPA formulation is provided, including the following active ingredients in parts by weight:

RAPA 1 to 100 parts,

phospholipid 1 to 2,000 parts, and

stabilizer 0.01 to 100 parts.

Further, the RAPA formulation includes 0.01 to 20,000 parts of a lyophilization protective agent.

Further, the lyophilization protective agent is at least one selected from the group consisting of lactose, glucose, mannitol, sucrose, and trehalose.

Further, the phospholipid is at least one selected from the group consisting of lecithin, cephalin, phosphatidylserine, phosphatidylglycerol (PG), phosphatidylinositol (PI), sphingomyelin, diphosphatidylglycerol (DPG), dipalmitoylphosphatidylcholine (DPPC), dioleoylphosphatidylethanolamine (DOPE), and distearoylphosphatidylethanolamine (DSPE).

Further, the lecithin is at least one selected from the group consisting of soy lecithin and hydrogenated soy lecithin.

Further, the stabilizer is at least one selected from the group consisting of cholesterol, sodium cholesterol sulfate, ethyl polyenoate, glycerol, and poloxamer.

The second objective of the present disclosure may be achieved through the following technical solutions: A preparation method of a RAPA formulation is provided, including:

mixing and dissolving RAPA, a phospholipid, and a stabilizer in an organic phase solvent to obtain an organic phase mixed solution;

preparing an initial emulsion solution by adding the organic phase mixed solution dropwise to an aqueous phase solvent, and stirring a resulting mixture at a temperature lower than or equal to 40° C. for 30 min to 150 min to obtain the initial emulsion solution; and

conducting lyophilization by homogenizing the initial emulsion solution, adding a lyophilization protective agent for mixing, and filtering a resulting mixture through a microporous filter membrane for sterilization to obtain the RAPA formulation of a liposome-lyophilized powder.

Further, in the lyophilization, the resulting mixture is filtered through a microporous filter membrane with a pore size of 0.22 μm to 0.45 μm for sterilization.

The third objective of the present disclosure may be achieved through the following technical solutions: A preparation method of a RAPA formulation is provided, including:

mixing and dissolving RAPA and a phospholipid in an organic phase solvent and removing a non-oil phase substance through rotary evaporation to obtain an initial mixed solution;

preparing an initial emulsion solution by adding a stabilizer to an aqueous phase solvent, adding the initial mixed solution, and stirring to obtain the initial emulsion solution; and

conducting pH adjustment by adjusting the pH of the initial emulsion solution to 8 to 9 and homogenizing to obtain the RAPA formulation of a fat emulsion.

Further, in the preparation of the initial emulsion solution, the stirring is conducted at a stirring speed of 300 rpm to 1,200 rpm.

Further, in the pH adjustment, the pH is adjusted with a 0.1 M NaOH solution, and the homogenization is conducted under a pressure of 300 bar to 1,000 bar.

Further, the organic phase solvent is at least one selected from the group consisting of absolute ethanol, dichloromethane (DCM), tertiary butyl alcohol (TBA), acetone, methanol, soybean oil, medium-chain triglyceride (MCT), and oleic acid.

Further, the aqueous phase solvent is at least one selected from the group consisting of distilled water, normal saline (NS), cell culture medium, body fluid, and buffer.

Compared with the prior art, the present disclosure has the following beneficial effects:

1. The RAPA formulation of the present disclosure can have prominent affinity and targetability for tumor cells and can improve an uptake rate of RAPA by a tumor tissue by targeting tumor cells, thus leading to the apoptosis of tumor cells, thereby treating the tumor.

2. An amount of the phospholipid in the RAPA preparation of the present disclosure is restricted, and the amount of the phospholipid affects the encapsulation rate of RAPA. A too-high phospholipid amount will lead to a waste of raw materials and a decrease in drug load. A too-low phospholipid amount will lead to incomplete encapsulation of RAPA. When the stabilizer is used at an amount in the defined range, the liposome can be the most stable and have the fewest side effects.

3. In the preparation method of the RAPA formulation in the present disclosure, RAPA is prepared into a liposome-lyophilized powder and a fat emulsion, and the formulations have a stable encapsulation rate and drug load, which can increase the concentration of RAPA in tumor cells and reduce the side effects and toxic effects of RAPA on normal cells.

4. In the preparation method of the RAPA formulation in the present disclosure, during the preparation of the initial emulsion solution, the temperature is controlled at 40° C. or lower. If the temperature is higher than 40° C., the phospholipid will be broken. The stirring time is controlled at 30 min to 150 min, and a stirring time that is too short will lead to non-thorough stirring, a liposome with a particle size that is too large, and an encapsulation rate that is too low. A stirring time that is too long will cause release of RAPA, resulting in the unsuccessful preparation of the liposome.

### BRIEF DESCRIPTION OF THE DRAWINGS

FIG. 1 shows the appearance of the formulations obtained in Examples 1 to 4;

FIG. 2 shows the appearance of the formulations obtained in Examples 1 to 4 after being dissolved;

FIG. 3 shows the particle simulation of a RAPA formulation of a liposome;

FIG. 4 shows a particle size distribution of a RAPA formulation;

FIG. 5 is a transmission electron microscopy (TEM) image of a RAPA formulation;

FIG. 6 is a zeta potential diagram of a RAPA formulation;

FIG. 7 shows an inhibitory effect of a RAPA formulation on cells;

FIG. 8 shows an uptake rate of a tumor cell for a RAPA formulation of a liposome;

FIGS. 9A-9B are schematic diagrams of clone colonies;

FIGS. 10A-10B are schematic diagrams of tumor cell apoptosis; and

FIGS. 11A-11B are schematic diagrams of tumor cell migration.

### DETAILED DESCRIPTION OF THE EMBODIMENTS

The present disclosure is further described below in combination with the accompanying drawings and specific implementations.

1) Preparation of a RAPA formulation of a liposome-lyophilized powder:

Specific steps are as follows:

Mixing: RAPA, a phospholipid, and a stabilizer are dissolved in an organic phase solvent to obtain an organic phase mixed solution.

Preparation of an initial emulsion solution: The organic phase mixed solution is added to an aqueous phase solvent at a speed of 1 to 10 drops/min The resulting mixture is stirred for 30 min to 150 min at a temperature lower than or equal to 40° C. and a stirring speed of 300 rpm to 1,200 rpm to obtain the initial emulsion solution. In the initial emulsion solution, a concentration of the RAPA is 1 to 100 mg/100 mL, a concentration of the phospholipid is 1 to 2,000 mg/100 mL, and a concentration of the stabilizer is 0.01 to 100 mg/100 mL.

Lyophilization: The initial emulsion solution is homogenized 5 to 20 times on a homogenizer under a homogenization pressure of 300 bar to 1,000 bar, and a lyophilization protective agent is added at a concentration of 0.01 to 20,000 mg/100 mL. The resulting mixture is filtered through a microporous filter membrane with a pore size of 0.22 μm to 0.45 μm for sterilization and then lyophilized or autoclaved to obtain the RAPA formulation of the liposome-lyophilized powder, which has an average particle size of 10 nm to 200 nm, a drug load of 1% to 40%, and an encapsulation rate of 85% or higher.

The lyophilization protective agent is at least one selected from the group consisting of lactose, glucose, mannitol, sucrose, and trehalose.

The formulation has high dispersibility and a large surface area, which is conducive to increasing the contact time and contact area between the drug and a biofilm at an absorption site and increasing the solubility of the drug. The formulation can enter cells through an endocytosis mechanism, and the endocytosis mechanism is different from a transmembrane transport mechanism of a general drug, which can increase the transmittance of the drug for a biofilm.

A liposome is a nano-scale carrier prepared with a lipid bilayer, and the lipid bilayer can encapsulate a lipid-soluble and water-soluble drug. In addition, a liposome has excellent biocompatibility and can be metabolized normally. A liposome is essentially a phospholipid substance and has a prominent affinity for tumor cells. Given the strong uptake ability of tumor cells for a liposome, the content of the drug in tumor cells can be increased, such that the drug is concentrated in tumor cells to treat a tumor. Compared with other drug-loading systems, liposomes have various advantages such as targetability, an affinity for tumor cells, prolonged drug action time, reduced drug toxicity, and protection for an encapsulated drug.

In the present disclosure, through the encapsulation of an amphiphilic phospholipid and the solubilization and dispersion of an organic phase solvent, a nanoparticle structure of the liposome is finally formed in an aqueous phase solvent. The nanoparticle structure of the liposome increases the solubility of RAPA in an aqueous phase and the uptake rate of tumor cells for RAPA, has a specified sustained release effect and targeting effect, and can improve the therapeutic effect of RAPA. The RAPA formulation provided by the present disclosure is a nanoliposome structure, which has a uniform and stable particle size distribution and a stable encapsulation rate and drug load and poses little risk to blood vessels. The RAPA formulation has a stable encapsulation rate and drug load and a prominent tumor-targeting effect. The RAPA preparation treats a tumor by inducing apoptosis of tumor cells.

2) Preparation of a RAPA formulation of a fat emulsion:

Specific steps are as follows:

Mixing: RAPA and a phospholipid are dissolved in an organic phase solvent, and a non-oil phase substance is removed through rotary evaporation to obtain an initial mixed solution.

Preparation of an initial emulsion solution: A stabilizer is added to an aqueous phase solvent, then the initial mixed solution is added, and a resulting mixture is stirred for 30 min at a stirring speed of 300 rpm to 1,200 rpm to obtain the initial emulsion solution. In the initial emulsion solution, a concentration of the RAPA is 1 to 100 mg/100 mL, a concentration of the phospholipid is 1 to 2,000 mg/100 mL, and a concentration of the stabilizer is 0.01 to 100 mg/100 mL.

pH adjustment: A pH of the initial emulsion solution is adjusted with a 0.1 M NaOH solution to 8 to 9, and then the initial emulsion solution is homogenized 3 to 10 times under a homogenization pressure of 300 bar to 1,000 bar to obtain the RAPA formulation of the fat emulsion, which has an average particle size of 10 nm to 1,000 nm, a drug load of 1% to 40%, and an encapsulation rate of 85% or higher.

Among the components for preparing the RAPA formulations of the liposome-lyophilized powder and the fat emulsion:

The phospholipid is at least one selected from the group consisting of lecithin, cephalin, phosphatidylserine, PG, PI, sphingomyelin, DPG, DPPC, DOPE, and DSPE. The lecithin can be hydrogenated soy lecithin, and the use of hydrogenated soy lecithin can increase the stability of a liposome.

The stabilizer is at least one selected from the group consisting of cholesterol, sodium cholesterol sulfate, ethyl polyenoate, glycerol, and poloxamer.

The organic phase solvent is at least one selected from the group consisting of absolute ethanol, DCM, TBA, acetone, methanol, soybean oil, MCT, and oleic acid.

The aqueous phase solvent is at least one selected from the group consisting of distilled water, NS, cell culture medium, body fluid, and buffer.

The RAPA formulations of the liposome-lyophilized powder and the fat emulsion each are used as follows: A liquid for injection, such as NS for injection, a glucose solution for injection, or a sugar-salt solution for injection, is added, and the resulting mixture is thoroughly mixed to obtain an injection solution in which the concentration of RAPA is generally 5 to 100 mg/100 mL.

### Example 1

Preparation of a RAPA Formulation of a Liposome-Lyophilized Powder:

Mixing: 5 mg of RAPA, 40 mg of hydrogenated soy lecithin, and 3.75 mg of cholesterol (stabilizer) were dissolved in 3 mL of DCM (organic phase solvent) to obtain an organic phase mixed solution.

Preparation of an initial emulsion solution: The organic phase mixed solution was added at a speed of 1 to 10 drops/min to 40 mL of phosphate-buffered saline (PBS) (aqueous phase solvent), and the resulting mixture was stirred for 60 min at a temperature of lower than or equal to 40° C. and a stirring speed of 600 rpm to obtain the initial emulsion solution.

Lyophilization: The initial emulsion solution was homogenized 6 times on a homogenizer under a homogenization pressure of 900 bar, and 2 g of lactose (lyophilization protective agent) was added, and the resulting mixture was mixed, filtered through a microporous filter membrane with a pore size of 0.22 μm for sterilization, and lyophilized to obtain the RAPA formulation of the liposome-lyophilized powder.

### Example 2

Preparation of a RAPA Formulation of a Liposome-Lyophilized Powder:

Mixing: 50 mg of RAPA, 450 mg of a phospholipid, and 50 mg of cholesterol (stabilizer) were dissolved in 10 mL of DCM (organic phase solvent) to obtain an organic phase mixed solution.

Preparation of an initial emulsion solution: The organic phase mixed solution was added at a speed of 1 to 10 drops/min to 100 mL of PBS (aqueous phase solvent), and the resulting mixture was stirred for 60 min at a temperature of lower than or equal to 40° C. and a stirring speed of 600 rpm to obtain the initial emulsion solution.

Lyophilization: The initial emulsion solution was homogenized 6 times on a homogenizer under a homogenization pressure of 900 bar, and 5 g of lactose (lyophilization protective agent) was added, and the resulting mixture was mixed, filtered through a microporous filter membrane with a pore size of 0.22 μm for sterilization, and lyophilized to obtain the RAPA formulation of the liposome-lyophilized powder.

### Example 3

Preparation of a RAPA formulation of a liposome-lyophilized powder:

Mixing: 50 mg of RAPA, 450 mg of a phospholipid, and 50 mg of cholesterol (stabilizer) were dissolved in 10 mL of DCM (organic phase solvent) to obtain an organic phase mixed solution.

Preparation of an initial emulsion solution: The organic phase mixed solution was added at a speed of 1 to 10 drops/min to 100 mL of PBS (aqueous phase solvent), and the resulting mixture was stirred for 60 min at a temperature of lower than or equal to 40° C. and a stirring speed of 600 rpm to obtain the initial emulsion solution.

Lyophilization: The initial emulsion solution was homogenized 6 times on a homogenizer under a homogenization pressure of 900 bar, and 5 g of trehalose (lyophilization protective agent) was added, and the resulting mixture was mixed, filtered through a microporous filter membrane with a pore size of 0.22 μm for sterilization, and lyophilized to obtain the RAPA formulation of the liposome-lyophilized powder.

### Example 4

Preparation of a RAPA Formulation of a Liposome-Lyophilized Powder:

Mixing: 240 mg of RAPA, 2,160 mg of hydrogenated soy lecithin, and 240 mg of cholesterol (stabilizer) were dissolved in 20 mL of DCM (organic phase solvent) to obtain an organic phase mixed solution.

Preparation of an initial emulsion solution: The organic phase mixed solution was added at a speed of 1 to 10 drops/min to 300 mL of distilled water (aqueous phase solvent), and the resulting mixture was stirred for 90 min at a temperature of lower than or equal to 40° C. and a stirring speed of 550 rpm to obtain the initial emulsion solution.

Lyophilization: The initial emulsion solution was homogenized 5 times on a homogenizer under a homogenization pressure of 600 bar, and 15 g of lactose (lyophilization protective agent) was added. The resulting mixture was mixed, filtered through a microporous filter membrane with a pore size of 0.22 μm for sterilization, and lyophilized to obtain the RAPA formulation of the liposome-lyophilized powder.

### Example 5

Preparation of a RAPA Formulation of a Liposome-Lyophilized Powder:

Mixing: 240 mg of RAPA, 2,160 mg of hydrogenated soy lecithin, and 240 mg of cholesterol (stabilizer) were dissolved in 20 mL of DCM (organic phase solvent) to obtain an organic phase mixed solution.

Preparation of an initial emulsion solution: The organic phase mixed solution was added at a speed of 1 to 10 drops/min to 300 mL of distilled water (aqueous phase solvent), and the resulting mixture was stirred for 90 min at a temperature of lower than or equal to 40° C. and a stirring speed of 550 rpm to obtain the initial emulsion solution.

Lyophilization: The initial emulsion solution was homogenized 5 times on a homogenizer under a homogenization pressure of 600 bar, and 15 g of trehalose (lyophilization protective agent) was added. The resulting mixture was mixed, filtered through a microporous filter membrane with a pore size of 0.22 μm for sterilization, and lyophilized to obtain the RAPA formulation of the liposome-lyophilized powder.

### Example 6

Preparation of a RAPA Formulation of a Liposome-Lyophilized Powder:

Mixing: 800 mg of RAPA, 7,200 mg of hydrogenated soy lecithin, and 800 mg of cholesterol (stabilizer) were dissolved in 100 mL of DCM (organic phase solvent) to obtain an organic phase mixed solution.

Preparation of an initial emulsion solution: The organic phase mixed solution was added at a speed of 1 to 10 drops/min to 1,000 mL of distilled water (aqueous phase solvent), and the resulting mixture was stirred for 90 min at a temperature of lower than or equal to 40° C. and a stirring speed of 600 rpm to obtain the initial emulsion solution.

Lyophilization: The initial emulsion solution was homogenized 6 times on a homogenizer under a homogenization pressure of 850 bar, and 50 g of trehalose (lyophilization protective agent) was added. The resulting mixture was mixed, filtered through a microporous filter membrane with a pore size of 0.22 μm for sterilization, and lyophilized to obtain the RAPA formulation of the liposome-lyophilized powder.

### Example 7

Preparation of a RAPA Formulation of a Fat Emulsion:

Specific steps were as follows:

Mixing: 10 mg of RAPA and 200 mg of hydrogenated soy lecithin (the hydrogenated soy lecithin was first dissolved with 2 mL of absolute ethanol) were dissolved with 1 g of MCT, 0.5 mL of oleic acid, and 1 mL of soybean oil (organic phase solvent). The non-oil phase substance (absolute ethanol) was then removed through rotary evaporation to obtain an initial mixed solution.

Preparation of an initial emulsion solution: 20 mg of Poloxamer 188 and 1 mL of glycerol (stabilizer) were added to 6.5 mL of distilled water (aqueous phase solvent) and heated to 60° C., and the initial mixed solution was preheated to 60° C. and added. The resulting mixture was stirred for 30 min at a stirring speed of 600 rpm to obtain the initial emulsion solution, and then 93.5 mL of distilled water (aqueous phase solvent) was added.

pH adjustment: The pH of the initial emulsion solution was adjusted with a 0.1 M NaOH solution to 8 to 9, and then the initial emulsion solution was homogenized 4 times under a homogenization pressure of 850 bar to obtain the RAPA formulation of the fat emulsion.

### Example 8

Preparation of a RAPA Formulation of a Fat Emulsion:

Specific steps were as follows:

Mixing: 30 mg of RAPA and 900 mg of hydrogenated soy lecithin (the hydrogenated soy lecithin was first dissolved with 2 mL of absolute ethanol) were dissolved with 7.5 g of MCT and 0.18 mL of oleic acid (organic phase solvent). The non-oil phase substance (absolute ethanol) was then removed through rotary evaporation to obtain an initial mixed solution.

Preparation of an initial emulsion solution: 12 mg of Poloxamer 188 and 0.9 mL of glycerol (stabilizer) were added to 21 mL of distilled water (aqueous phase solvent) and heated to 60° C., and the initial mixed solution was preheated to 60° C. and added. The resulting mixture was stirred for 30 min at a stirring speed of 600 rpm to obtain the initial emulsion solution, and then 79 mL of distilled water (aqueous phase solvent) was added.

pH adjustment: The pH of the initial emulsion solution was adjusted with a 0.1 M NaOH solution to 8 to 9, and then the initial emulsion solution was homogenized 10 times under a homogenization pressure of 400 bar to obtain the RAPA formulation of the fat emulsion.

### Example 9

Preparation of a RAPA Formulation of a Fat Emulsion:

Specific steps were as follows:

Mixing: 30 mg of RAPA and 900 mg of hydrogenated soy lecithin (the hydrogenated soy lecithin was first dissolved with 2 mL of absolute ethanol) were dissolved with 3.5 g of MCT, 0.18 mL of oleic acid, and 3.5 mL of soybean oil (organic phase solvent). The non-oil phase substance (absolute ethanol) was then removed through rotary evaporation to obtain an initial mixed solution.

Preparation of an initial emulsion solution: 12 mg of Poloxamer 188 and 0.9 mL of glycerol (stabilizer) were added to 21 mL of distilled water (aqueous phase solvent) and heated to 60° C., and the initial mixed solution was preheated to 60° C. and added. The resulting mixture was stirred for 30 min at a stirring speed of 600 rpm to obtain the initial emulsion solution, and then 79 mL of distilled water (aqueous phase solvent) was added.

pH adjustment: The pH of the initial emulsion solution was adjusted with a 0.1 M NaOH solution to 8 to 9, and then the initial emulsion solution was homogenized 10 times under a homogenization pressure of 400 bar to obtain the RAPA formulation of the fat emulsion.

Test:

1) Appearance evaluation and determination of average particle size, potential, and encapsulation rate

Each of the RAPA formulations obtained in the examples was evaluated for appearance and tested for average particle size, potential, encapsulation rate, peroxide value, and organic solvent residue.

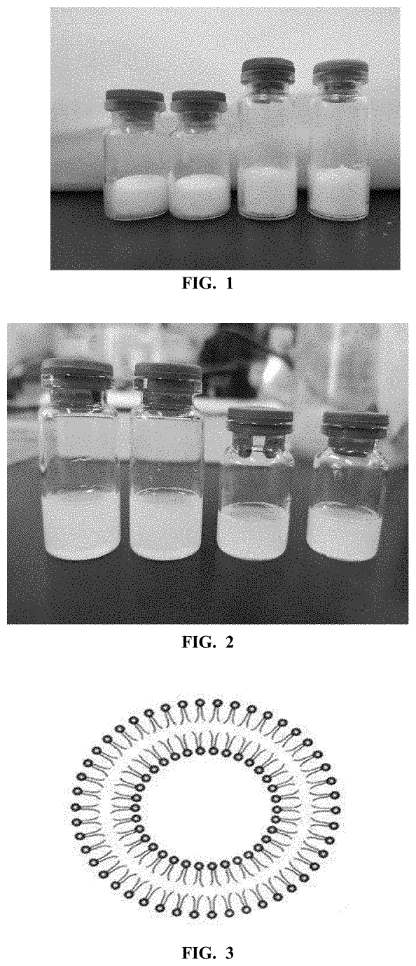

Appearance evaluation criteria: The following characteristics were desired: original volume, no collapse, no shrinkage, uniform color, no spot, and delicate texture, as shown in FIG. 1. After being dissolved, the formulation is not separated, as shown in FIG. 2. The appearance of the formulations in Examples 1 to 4 was shown from left to right in FIG. 1 and from right to left FIG. 2.

Average particle size: A Malvern laser particle size analyzer was used to determine the particle size and particle size distribution of nanoparticles. The particle size was determined according to the following principle: When particles are irradiated by light, light scattering and light diffraction occur, and the scattering intensity and diffraction intensity of light are both related to particle sizes and optical characteristics.

Peroxide value: According to the detection method for peroxide value in “Chinese Pharmacopoeia” (2015 edition), peroxide values in the liposomes of Examples 1, 3, and 5 were determined to be 0.86 meq/kg, 0.85 meq/kg, and 0.86 meq/kg, respectively, which met the requirements in “Chinese Pharmacopoeia”.

Organic solvent residue: According to the detection method for organic solvent residue in “Chinese Pharmacopoeia” (2015 edition), an organic solvent residue in Example 4 was determined to be 0.055%, which met the requirements in “Chinese Pharmacopoeia”.

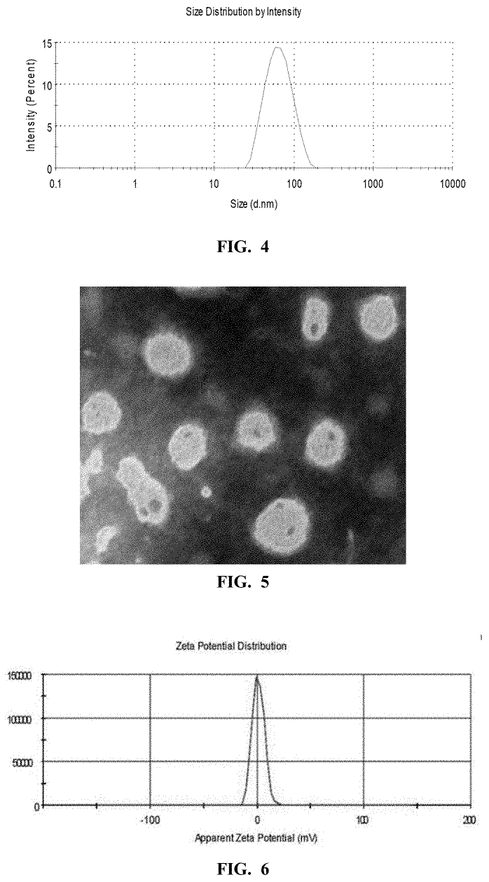

FIG. 3 shows the simulation of the RAPA formulation of a liposome in Example 4, where globular portions represent the active ingredient RAPA. FIG. 4 shows a particle size distribution of a RAPA formulation. FIG. 5 is a TEM image of a RAPA formulation.

Potential: A Malvern laser particle size analyzer was used to determine the potential of nanoparticles. FIG. 6 is a zeta potential diagram of a RAPA formulation.

The total content of the drug was determined with reference to a content determination method.

The drug content was determined by high-performance liquid chromatography (HPLC) with methanol-acetonitrile-water (in a volume ratio of 43:40:17) as a mobile phase, a flow rate of 1 mL/min, a column temperature of 40° C., and a detection wavelength of 278 nm.

The calculation formula for the encapsulation rate: encapsulation rate=encapsulated drug amount/total main drug content×100%

TABLE 1
Redispersibility, encapsulation rate, and average particle
size for the formulations in Examples 1 to 9

| | | | | Average |
|---|---|---|---|---|
| | | | Encapsulation | particle |
| Example | Appearance | Redispersibility | rate (%) | size (nm) |
| 1 | No shrinkage | Excellent | 89 | 105 |
| | and no collapse | | | |
| 2 | No shrinkage | Excellent | 86 | 109 |
| | and no collapse | | | |
| 3 | No shrinkage | Excellent | 94 | 110 |
| | and no collapse | | | |
| 4 | No shrinkage | Excellent | 92 | 102 |
| | and no collapse | | | |
| 5 | No shrinkage | Excellent | 95 | 106 |
| | and no collapse | | | |
| 6 | No separation | Excellent | 94 | 290 |
| 7 | No separation | Excellent | 92 | 273 |
| 8 | No separation | Excellent | 91 | 230 |
| 9 | No separation | Excellent | 92 | 260 |

It can be seen from Table 1 that the RAPA formulation obtained in the present disclosure has an encapsulation rate of 85% or higher.

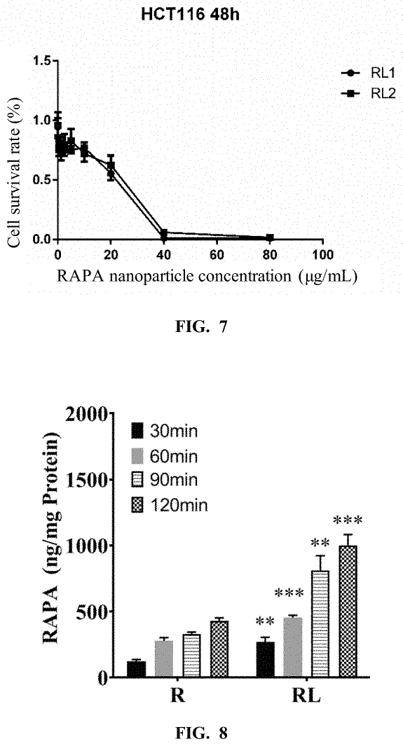

2) An MTT kit method was used to conduct a cytotoxicity experiment with HCT116 cells. The HCT116 cells were inoculated at 1×10⁴ cells/well into a 96-well plate, then cultivated in a 5% CO₂/37° C. incubator for 24 h, and then treated for 48 h with each of the RAPA formulations in Example 4 (RL1) and Example 8 (RL2) at concentrations (based on the active ingredient RAPA) of 80 μg/mL, 40.00 μg/mL, 30.00 μg/mL, 20.00 μg/mL, 10.00 μg/mL, 5.00 μg/mL, 2.50 μg/mL, 1.25 μg/mL, 0.65 μg/mL, 0.3125 μg/mL, and 0 μg/mL. As shown in FIG. 7, the RAPA formulations significantly inhibited the growth of the HCT116 cells, and after the 48 h of administration, an IC₅₀ value was 11.68 μg/mL.

3) Cell uptake experiment for the RAPA formulation of a liposome

2 mL of HCT116 cells in a logarithmic growth phase was inoculated into each well of a six-well plate at a cell density of about 2×10⁴ cells/well and then cultivated for 48 h in a 37° C./5% CO₂ incubator with saturated humidity. The cells were washed 3 times with PBS, 1% fetal bovine serum (FBS)-containing DMEM cell culture media with RAPA (R group) at 8 μg/mL and the RAPA formulation of a liposome (RL group) in Example 5 at 8 μg/mL each were added to the cells, and the cells were incubated for 30 min, 60 min, 90 min, and 120 min. Cell lysis was conducted, and a lysate was collected to determine an intracellular protein concentration in each well according to the BCA method. The uptake of the HCT116 cells for RAPA was determined by the liquid chromatography-mass spectrometry (LC-MS) method. Ion pairs for MS: RAPA: 936.47/936.47 (CE: 11 V; Tube Lens Voltage: 96.54 V), and internal standard danazol: 338.32/338.32 (CE: 15 V; Tube Lens Voltage: 106.11 V). Chromatographic resolution conditions: 0 min to 2 min: 80% methanol; 2 min to 3 min: 95% methanol; 3 min to 6 min: 95% methanol; 6 min to 7 min: 80% methanol; resolution time: 10 min; injection volume: 10 μL; and chromatographic column: Agilent SB-C18 2.1×100 nm 3.5 μm. Results are shown in FIG. 8. At different time points, the uptake rate of tumor cells for the RAPA formulation of a liposome was much higher than that for the active ingredient RAPA, indicating that the RAPA formulation of a liposome had strong affinity and targetability for tumor cells. Thus, the performance of the nano-formulation was far better than the performance of the active ingredient RAPA.

4) In vitro tumor inhibitory effect of the RAPA formulation of a liposome

4.1 Plate cloning experiment for detecting the proliferation of tumor cells

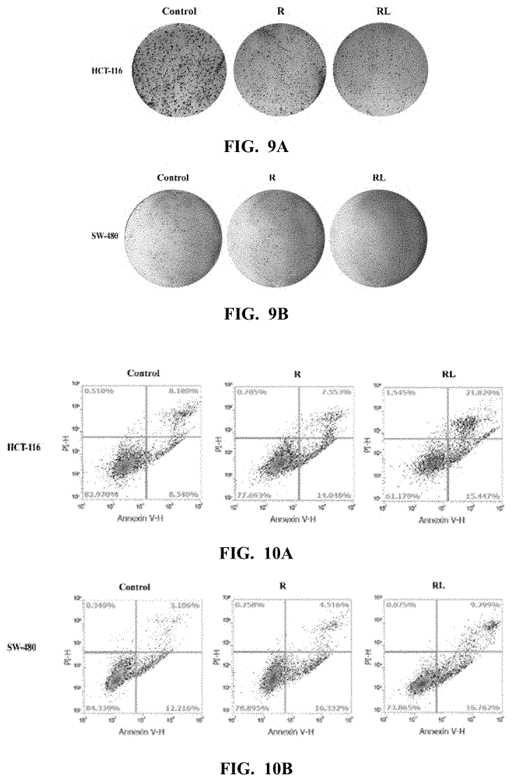

Each of the HCT116 and SW-480 cells in a logarithmic growth phase was prepared into a suspension with a 10% FBS-containing cell culture medium, blown into single cells, counted, inoculated into a six-well plate at a density of 1×10³ cells/well, and cultivated in a 5% CO₂/37° C. incubator with saturated humidity until the cells were adherent to the wall. The following three groups were set in this experiment: untreated group (Control group), free RAPA group (R group), and liposomal RAPA group (RL group, using the formulation in Example 3). After the administration, the cells were often observed, and when clone spheres visible to the naked eye appeared, the cell cultivation was stopped, the supernatant was discarded, the cells were washed twice with PBS, and 1 mL of 4% paraformaldehyde (PFA) was added to conduct fixation for 20 min. The fixation solution was removed, the cells were washed 3 times with PBS, a 1% gentian violet staining solution was added to conduct staining for 30 min, and then the cells were slowly rinsed with running water to remove the staining solution, dried in an oven at 37° C., and photographed and counted. Colony-forming efficiency (CFE)=(number of cell clones/number of inoculated cells)×100%. Results showed that the liposomal RAPA group significantly reduced the colony numbers for human colon cancer cells HCT116 and SW-480 compared with the untreated group. The colony number was calculated for each group. As shown in FIGS. 9A-9B, the colony numbers for HCT116 cells in the three groups were 62.0±7.6, 49.2±5.2, and 33.1±7.3, respectively, where the colony number in the liposomal RAPA group was significantly smaller than the colony number in the free RAPA group (p<0.01). The colony numbers for SW-480 cells in the three groups were 36.1±7.5, 33.2±3.7, and 22.5±4.6, respectively, where the colony number in the liposomal RAPA group was significantly smaller than the colony number in the free RAPA group (p<0.01).

4.2 Detection of apoptosis of tumor cells by flow cytometry (FCM)

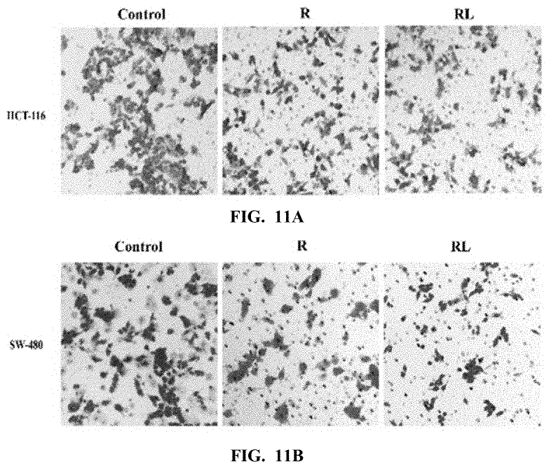

An apoptosis detection kit was used to detect the apoptosis, and the following three groups were set in this experiment: untreated group (Control group), free RAPA group (R group), and liposomal RAPA group (RL group, using the formulation in Example 1). 48 h after administration, a supernatant in each group was removed, the cells were washed with 1×PBS 3 times and then digested with EDTA-free trypsin for 2 min, and a 10% FBS-containing cell culture medium was added to stop the digestion. The resulting suspension was collected in a centrifuge tube and centrifuged at 1,000 rpm for 3 min, and resulting cells were washed twice with 1×PBS (4° C.) and resuspended in 400 μL of an Annexin V binding solution. 5 μL of an Annexin V-FITC staining solution was added, and the cells were incubated in the dark for 15 min. 10 μL of a PI staining solution was added, and the cells were incubated in the dark for 5 min. The cells were immediately tested by FC. Results showed that apoptosis rates for the HCT116 cells in the untreated group, the free RAPA group, and the liposomal RAPA group were 16%, 21%, and 37%, respectively. Compared with the untreated group, the apoptosis rate in the liposomal RAPA group was significantly increased (p<0.05). The results are shown in FIGS. 10A-10B.

4.3 Migration experiment to detect the migration ability of tumor cells

Each of the HCT116 and SW-480 cells in a logarithmic growth stage was taken and prepared into a suspension with a serum-free cell culture medium, counted, and diluted in folds, and a serum-free cell culture medium with the drug of 15 μg/mL was added. 200 μL of the suspension was inoculated into an upper chamber of a Transwell cell at a density of 1×10⁵ cells/well, 500 μL of a 10% FBS-containing cell culture medium was added to a lower chamber of the Transwell cell, and the Transwell cell was incubated in a 5% CO₂/37° C. incubator with saturated humidity for 36 h. The number of cells passing through the cell was counted, and cells in the upper chamber were gently wiped off with a cotton swab. Cells in the lower chamber were fixed with formaldehyde for 15 min, then stained with a 1% gentian violet staining solution for 30 min, and observed and counted under a microscope. As shown in FIGS. 11A-11B, for the HCT116 cells, percentages of cells passing through the cell in the untreated group (Control group), the free RAPA group (R group), and the liposomal RAPA group (RL group) were 100.0±12.5, 77.6±10.9, and 64.7±8.3, respectively, where a percentage of cells passing through the cell in the liposomal RAPA group was significantly smaller than a percentage of cells passing through the cell in the free RAPA group (p<0.01). For the SW-480 cells, percentages of cells passing through the cell in the untreated group, the free RAPA group, and the liposomal RAPA group were 100.0±11.7, 57.4±10.6, and 42.9±12.3, respectively, where a percentage of cells passing through the cell in the liposomal RAPA group was significantly smaller than a percentage of cells passing through the cell in the free RAPA group (p<0.05).

It can be seen from FIGS. 11A-11B that the formulation of the present disclosure can promote the apoptosis of tumor cells and significantly inhibit the proliferation and migration of tumor cells.

Corresponding changes and variations may be made by those skilled in the art according to the technical solutions and concepts described above, and all these changes and variations should fall within the protection scope of the claims of the present disclosure.

What is claimed is:

1. A rapamycin (RAPA) formulation, comprising the following active ingredients in parts by weight:
RAPA 1 to 100 parts;
a phospholipid 1 to 2,000 parts; and
a stabilizer 0.01 to 100 parts.

2. The RAPA formulation according to claim 1, further comprising: 0.01 to 20,000 parts of a lyophilization protective agent.

3. The RAPA formulation according to claim 2, wherein the lyophilization protective agent is at least one selected from the group consisting of lactose, glucose, mannitol, sucrose, and trehalose.

4. The RAPA formulation according to claim 1, wherein the phospholipid is at least one selected from the group consisting of lecithin, cephalin, phosphatidylserine, phosphatidylglycerol (PG), phosphatidylinositol (PI), sphingomyelin, diphosphatidylglycerol (DPG), dipalmitoylphosphatidylcholine (DPPC), dioleoylphosphatidylethanolamine (DOPE), and distearoylphosphatidylethanolamine (DSPE).

5. The RAPA formulation according to claim 1, wherein the stabilizer is at least one selected from the group consisting of cholesterol, sodium cholesterol sulfate, ethyl polyenoate, glycerol, and poloxamer.

6. A preparation method of a RAPA formulation, comprising:
mixing and dissolving RAPA, a phospholipid, and a stabilizer in an organic phase solvent to obtain an organic phase mixed solution;
preparing an initial emulsion solution by adding the organic phase mixed solution dropwise to an aqueous phase solvent to obtain a first resulting mixture, and stirring the first resulting mixture at a temperature lower than or equal to 40° C. for 30 min to 150 min to obtain the initial emulsion solution; and
conducting a lyophilization by homogenizing the initial emulsion solution, adding a lyophilization protective agent for mixing to obtain a second resulting mixture, and filtering the second resulting mixture through a microporous filter membrane for a sterilization to obtain the RAPA formulation of a liposome-lyophilized powder.

7. The preparation method of the RAPA formulation according to claim 6, wherein in the lyophilization, the second resulting mixture is filtered through the microporous filter membrane with a pore size of 0.22 μm to 0.45 μm for the sterilization.

8. A preparation method of a RAPA formulation, comprising:
mixing and dissolving RAPA and a phospholipid in an organic phase solvent, and removing a non-oil phase substance through a rotary evaporation to obtain an initial mixed solution;
preparing an initial emulsion solution by adding a stabilizer to an aqueous phase solvent to obtain a first resulting mixture, adding the initial mixed solution to the first resulting mixture to obtain a second resulting mixture, and stirring the second resulting mixture to obtain the initial emulsion solution; and
conducting a pH adjustment by adjusting a pH of the initial emulsion solution to 8 to 9 and homogenizing the initial emulsion solution to obtain the RAPA formulation of a fat emulsion.

9. The preparation method of the RAPA formulation according to claim 8, wherein in a preparation of the initial emulsion solution, the stirring is conducted at a stirring speed of 300 rpm to 1,200 rpm.

10. The preparation method of the RAPA formulation according to claim 8, wherein in the pH adjustment, the pH is adjusted with a 0.1 M NaOH solution; and the homogenizing is conducted under a pressure of 300 bar to 1,000 bar.
